(19) United States
(12) Patent Application Publication
(10) Pub. No.: US 2006/0147383 A1
(43) Pub. Date: Jul. 6, 2006

(54) Sprayable compositions comprising pharmaceutical active agents, volatile silicones and a non-volatile oily phase

(75) Inventors: Claire Mallard, Mougins Le Haut (FR); Franck Pitre, Bussy Saint Georges (FR); Laurent Fredon, Roquefort Les Pins (FR)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(73) Assignee: GALDERMA RESEARCH & DEVELOPMENT, S.N.C., VALBONNE SOPHIA ANTIPOLIS (FR)

(21) Appl. No.: 11/315,259

(22) Filed: Dec. 23, 2005

Related U.S. Application Data
(63) Continuation of application No. PCT/EP04/07203, filed on Jun. 18, 2004.

(30) Foreign Application Priority Data
Jun. 23, 2003 (FR) 03/07551

(51) Int. Cl. A61K 31/573; A61L 9/04

(52) U.S. Cl. 424/45; 514/179

(57) ABSTRACT

Compositions in the form of a spray contain a pharmaceutical active agent, at least one volatile silicone and a non-volatile oily phase, formulated into a physiologically acceptable medium, are well suited for improved transdermal administration in cosmetics and dermatology.

### CROSS-REFERENCE TO PRIORITY/PCT APPLICATIONS

This application claims priority under 35 U.S.C. § 119 of FR 03/07551, filed Jun. 23, 2003, and is a continuation of PCT/EP 2004/007203, filed Jun. 18, 2004 and designating the United States (published in the French language on Dec. 29, 2004 as WO 2004/112798 A1; the title and abstract were also published in English), each hereby expressly incorporated by reference and each assigned to the assignee hereof.

### BACKGROUND OF THE INVENTION

1. Technical Field of the Invention

The invention relates to compositions comprising a pharmaceutical active agent, at least one volatile silicone and a non-volatile oily phase formulated into a physiologically acceptable medium therefor, to the process for preparing same and to their applications in cosmetics and in dermatology, the subject compositions enabling good penetration of the active agent through the layers of the skin.

2. Description of Background and/or Related and/or Prior Art

In the fields of dermatology and of the formulation of pharmaceutical compositions, those skilled in the art seek compositions which make it possible to release the active agent and to promote its penetration through the layers of the skin in order to improve its effectiveness. The product should also show good cosmeticity and preferably be non-irritant.

There currently exist many topical compositions comprising an active agent and making it possible to promote penetration thereof into the skin by means of the presence, in particular, of a high content of pro-penetrating glycol. These compositions are formulated in the form of emulsions with a high content of fatty phase, which are commonly called “lipocreams”, in the form of anhydrous compositions which are called “ointments”, in the form of fluid compositions with a high content of volatile solvents, such as ethanol or isopropanol, intended for application to the scalp, also called “hair lotions”, or else in the form of viscous O/W emulsions, which are also called “O/W creams”.

O/W creams comprising a corticoid and a high percentage of propylene glycol (47.5%), sold under the trademark TEMOVATE® by GLAXOSMITHKLINE, are, for example, known. The stabilizing of a formulation comprising such a percentage of glycol makes it necessary to include, in the emulsion, emulsifiers and stabilizers of the glyceryl stearate or PEG 100 stearate type, or alternatively stabilizers or consistency factors of the white wax or cetostearyl alcohol type, which result in the formation of a viscous cream, that is to say a cream with a viscosity greater than 10 Pa·s (10,000 centipoises, measured with a Brookfield model LVDV II+mobile No. 4 device, at a rate of 30 rpm for 30 seconds and at a temperature of 25° C.±3° C.). This viscosity therefore makes the product difficult to apply. These compositions therefore show, firstly, poor cosmetic acceptability due to their viscosity and, secondly, risks of intolerance caused by the presence of high proportions of glycol. Those skilled in the art therefore wish to improve these parameters.

In order to facilitate the application of topical compositions comprising a high percentage of pro-penetrating glycol, the assignee hereof has developed and described in EP-832,647, a lotion, which is a stable formulation of O/W emulsion type, and the viscosity of which is intermediate between hair lotions which are too fluid and have too limited a use, and O/W creams which are too viscous and have a greasy and sticky side to them, while at the same time conserving the pro-penetrating properties of the glycol. These formulae effectively show good penetration of the active agent, but still comprise a high percentage of glycol which can therefore induce a sticky effect or problems of tolerance resulting in moderate acceptability of the product by the patient.

Formulations containing silicone compounds which result in compositions which are pleasant to use are, moreover, known to those skilled in the art. Thus, in U.S. Pat. No. 6,538,039, a novel formulation of active agent for transdermal administration has been developed, comprising silicone compounds in order to deposit a film at the surface of the skin. In that application also, the transdermal passage is facilitated by the obligatory presence of absorption promoters, namely, among other compounds mentioned, glycols.

In EP-0-966,972, the compositions described can be formulated in the form of a spray and comprise an active compound, a silicone gum and a pharmaceutically acceptable excipient. The problem that the invention described in EP-0-966,972 proposes to solve is that of depositing a substantive film at the surface of the skin, which problem is solved by means of the presence of the silicone gum.

The problem that the present invention here proposes to solve is that of designing a composition for improving the penetration of the pharmaceutical active agent, and its rapidity of penetration over time, in order to improve its therapeutic efficacy, while at the same time avoiding the presence of a high content of glycol. The compositions according to the invention should also be easy to use and show a cosmeticity which is acceptable for application to all the regions of the body which may be affected by the pathology.

EP-0-966,972 and U.S. Pat. No. 6,538,039 represent the prior art closest to the present invention, given the composition of the formulations described. However, on reading this prior art, there is nothing which could prompt those skilled in the art to choose the compositions according to the invention in order to obtain good penetration of the active agent incorporated, into the layers of the skin.

### SUMMARY OF THE INVENTION

It has now surprisingly been found that compositions comprising, formulated into a pharmaceutically acceptable alcoholic vehicle:
a) a therapeutically effective amount of a pharmaceutical active agent,
b) at least one volatile silicone, and
c) a non-volatile oily phase,
   provide an improvement in penetration of the active agent.

The compositions of the present invention, while allowing good penetration of the active principles, also shows very good acceptability and tolerance among patients, as described in Examples 8 and 9 to follow. It is therefore found that the compositions according to the invention are particularly suitable for the treatment of dermatological conditions and afflictions, and more particularly very suitable for the treatment of psoriasis.

This invention therefore features sprayable compositions comprising, formulated into a pharmaceutically acceptable alcoholic vehicle:
a) a therapeutically effective amount of at least one pharmaceutical active agent,
b) at least one volatile silicone, and
c) a non-volatile oily phase.

### DETAILED DESCRIPTION OF BEST MODE AND SPECIFIC/PREFERRED EMBODIMENTS OF THE INVENTION

Among the pharmaceutical active agents according to the invention, mention may be made, by way of example, of agents for modulating the differentiation and/or proliferation and/or pigmentation of the skin, such as retinoic acid and isomers thereof, retinol and esters thereof, retinal, retinoids, in particular those described in FR-2-570,377, EP-1 99,636, EP-325,540 and EP-402,072, vitamin D and derivatives thereof, estrogens such as estradiol, kojic acid or hydroquinone; anti-bacterial agents such as clindamycin phosphate, erythromycin or antibiotics of the tetracycline class; anti-parasitic agents, in particular metronidazole, crotamiton or pyrethrinoids; anti-fungal agents, in particular compounds belonging to the imidazole class, such as econazole, ketoconazole or miconazole, or salts and derivatives thereof; polyene compounds, such as amphotericin B; compounds of the allylamine family, such as terbinafine; compounds of the pyridinone family, such as cyclopirox; compounds of the morpholine family and derivatives, such as amorolfine; steroidal anti-inflammatories, such as hydrocortisone, anthralins (dioxyanthranol), anthranoids, betamethasone valerate or clobetasol 17-propionate, or non-steroidal anti-inflammatories, such as ibuprofen and salts or derivatives thereof, diclofenac and salts and derivatives thereof, acetylsalicylic acid, acetaminophen or glycyrrhetinic acid; anaesthetics such as lidocaine hydrochloride and derivatives thereof; anti-pruriginous agents such as thenaldine, trimeprazine or cyproheptadine; anti-viral agents such as acyclovir; keratolytic agents such as alpha- and beta-hydroxycarboxylic acids or beta-ketocarboxylic acids, salts, amides or esters thereof, and more particularly hydroxy acids such as glycolic acid, lactic acid, malic acid, salicylic acid, citric acid and fruit acids in general, and 5-n-octanoyl-salicylic acid; free-radical scavengers, such alpha-tocopherol or esters thereof, superoxide dismutases, certain metal-chelating agents or ascorbic acid and esters thereof; anti-seborrhoeic agents such as progesterone; anti-dandruff agents such as octopirox or zinc pyrithione; anti-acne agents such as retinoic acid, benzoyl peroxide or adapalene; anti-metabolites; agents for combating hair loss, such as minoxidil; antiseptics; hormones or peptides.

The active agents according to the invention may be employed alone or in combination.

Advantageously, the compositions according to the invention comprise from 0.0001 to 20% by weight, relative to the total weight of the composition, of an active agent, preferably from 0.025 to 15% by weight, and more preferably from 0.01 to 5% by weight.

Of course, the amount of active agent in the compositions according to the invention will depend on the active agent under consideration.

The compositions according to the invention will preferably comprise a steroidal anti-inflammatory of corticoid type, in particular clobetasol 17-propionate at a concentration of less than 2%, and preferably from 0.01 to 2% by weight of active agent, more preferably from 0.025 to 0.1% by weight. The preferred pharmaceutical active agent according to the invention is clobetasol 17-propionate, at a concentration of 0.05% by weight.

According to the invention, the term “volatile silicone” means polyorganosiloxane compounds, which may be cyclic or linear, having a measurable pressure under ambient conditions.

The cyclic volatile silicones according to the invention are polydimethylcyclosiloxanes, i.e., compounds having the formula: 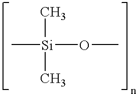
with n ranging, on average, from 3 to 6, and preferably n=4 or n=5, generally known as cyclomethicones.

The linear volatile silicones according to the invention are low molecular weight linear polysiloxanes such as hexamethyldisiloxane or low molecular weight dimethicones. The linear volatile silicones generally have a viscosity of less than approximately 5 centistokes at 25° C., whereas the cyclic volatile silicones have a viscosity of less than approximately 10 centistokes at 25° C.

Preferred volatile silicones according to the invention are the linear siloxanes, and more preferably hexamethyldisiloxane. By way of example, mention may be made of the product sold by DOW CORNING, DC Fluid 0.65 cSt.

Advantageously, the compositions according to the invention comprise from 25 to 95% by weight, relative to the total weight of the composition, of the volatile silicone, and preferably from 40 to 80% by weight, and more preferably from 55 to 65% by weight.

According to the invention, the term “non-volatile oily phase” means a variety of non-volatile oil suitable for a pharmaceutical or cosmetic composition. The non-volatile oils generally have a viscosity of greater than approximately 10 centipoises at 25° C., and can reach a viscosity ranging up to 1,000,000 centipoises at 25° C. The non-volatile oily phase can be made up of a large variety of synthetic or natural, silicone or organic oils, a non-exhaustive list of which now follows by way of example.

(a) Esters:

Examples of a non-volatile oil which can be incorporated according to the invention comprise esters of formula RCO—OR′ with R and R′, which may be identical or different, representing a linear or branched chain of an alkyl, alkenyl, alkoxycarbonylalkyl or alkoxycarbonyloxyalkyl radical having from 1 to 25 carbon atoms, preferably from 4 to 20 carbon atoms. Examples of such esters include isotridecyl isononanoate, PEG-4 diheptanoate, isostearyl neopentanoate, tridecyl neopentanoate, cetyl octanoate, cetyl palmitate, cetyl ricinoleate, cetyl stearate, cetyl myristate, coco dicaprylate/caprate, decyl isostearate, isodecyl oleate, isodecyl neopentanoate, isohexyl neopentanoate, octyl palmitate, dioctyl malate, tridecyl octanoate, myristyl myristate and octododecanol.

(b) Glyceryl Esters of Fatty Acids:

The oil may also comprise fatty esters of natural fatty acids, or triglycerides of animal or plant origin. Such examples include, castor oil, lanolin oil, triisocetyl citrate, triglycerides having from 10 to 18 carbon atoms, caprylic/capric triglycerides, coconut oil, corn oil, cottonseed oil, flax oil, mink oil, olive oil, palm oil, illipe butter, rapeseed oil, soybean oil, sunflower oil, nut oil and equivalent.

(c) Fatty Acid Glycerides:

The oils which are also suitable are synthetic or semi-synthetic glyceryl esters, such as fatty acid mono-, di or triglycerides, which are modified natural oils or fats, for example glyceryl stearate, glyceryl dioleate, glyceryl distearate, glyceryl trioctanoate, glyceryl distearate, glyceryl linoleate, glyceryl myristate, glyceryl isostearate, PEG castor oils, PEG glyceryl oleates, PEG glyceryl stearates, and equivalent.

(d) Non-Volatile Hydrocarbons:

Non-volatile hydrocarbons such as paraffins, isoparaffins, mineral oils, and equivalent are also very suitable for the compositions according to the invention, as the non-volatile oily phase.

(e) Guerbet Esters:

Guerbet esters are esters resulting from the reaction of a Guerbet alcohol of general formula: 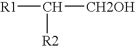
and a carboxylic acid of general formula R3—COOH or HOOC—R3—COOH, in which R1 and R2, which may be identical or different, represent an alkyl radical having from 4 to 20 carbon atoms, and R3 represents a substituted or unsubstituted fatty radical, such as a linear or branched, saturated or unsaturated alkyl or alkylene chain having from 1 to 50 carbon atoms, a phenyl, which may be substituted with a halogen, a hydroxyl, a carboxyl, or an alkylcarbonylhydroxyl.

(f) Silicone Oils:

The silicone oils according to the invention for constituting the non-volatile phase are polyorganosiloxane compounds having a measurable pressure under ambient conditions and a viscosity strictly greater than 10 centistokes. The non-volatile silicones according to the invention are the compounds of formula: 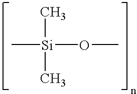
with n strictly greater than 6.

The preferred non-volatile oily phase according to the invention is paraffin oil.

Advantageously, the compositions according to the invention comprise from 1 to 50% by weight, relative to the total weight of the composition, of non-volatile oily phase, preferably from 5 to 30% by weight, and more preferably from 5 to 15% by weight.

Thus, a preferred composition according to the invention will be a sprayable composition comprising:
a) from 0.0001 to 20% by weight of the pharmaceutical active agent,
b) from 25 to 95% by weight of volatile silicone, and
c) from 1 to 50% by weight of non-volatile oily phase.

According to a preferred embodiment of a composition according to the invention, the composition also comprises a silicone gum. It has indeed surprisingly now been determined that a composition comprising a silicone gum in the concentrations defined hereinafter shows more rapid penetration of the active agent through the various layers of the skin.

The term “silicone gums” means the silicone gums known to those skilled in the art, and in particular those described in EP-0-966,972, incorporated herein by way of reference. According to this preferred embodiment of a composition according to the invention, the silicone gum is introduced at a concentration from 0.001 to 3% by weight, preferably from 0.01 to 1% by weight. Dow Corning provides a commercial product sold under the name DC Silmogen Carrier, which is made up of 99% of hexamethyldisiloxane and 1% of silicone gum, which product may advantageously be used in one of the compositions according to the invention.

The pharmaceutically acceptable vehicle according to the invention should be selected such that the advantageous properties intrinsically associated with the present invention are not, or are not substantially, altered by the envisaged addition. Preferably, the vehicle according to the invention is selected so as to be an agent which solubilizes the active agent. The active agent-solubilizing vehicle may be made up of a single excipient, such as a solvent, or of a mixture of excipients, such as those used for the formulation of an emulsion. By way of non-limiting examples of excipients which may be used alone or as a mixture, mention may be made of water, solvents, diluents, and any excipient which can be used for the formulation of an emulsion, of a milk, of a gel, of an ointment, or of a foaming composition. These excipients are compounds commonly used in the formulation of a pharmaceutical composition. Preferably, the active agent-solubilizing excipients according to the invention are water, alcohols, polyols, ethers, esters, aldehydes, ketones, fatty acids and fatty alcohols, and fatty esters. More preferably, the excipient used will be an alcohol. According to the invention, the term “alcohol” means linear or branched aliphatic alcohols such as ethanol, propanol or isopropanol.

In a preferred embodiment according to the invention, the vehicle used will therefore be alcoholic.

According to the invention, the term “alcoholic vehicle” means a vehicle comprising at least 15% of alcohol, and preferably at least 25% of ethanol.

In particular, the composition according to the invention as described above will be such that it contains:
a) 0.05% of clobetasol 17-propionate,
b) 60% of hexamethyldisiloxane,
c) 10% of paraffin oil,
d) 29.95% of ethanol.

More particularly, the composition according to the invention will be such that it comprises:
a) 0.05% of clobetasol 17-propionate,
b) 59.4% of hexamethyldisiloxane,
c) 0.6% of silicone gum,
d) 10% of paraffin oil,
e) 29.95% of ethanol.

The pharmaceutical composition according to the invention may also contain inert additives or combinations of these additives, such as:
wetting agents;
flavor enhancers;
preservatives, such as para-hydroxybenzoic acid esters;
stabilizers;
moisture regulators;
pH regulators;
osmotic pressure modifiers;
emulsifiers;
UV-A and UV-B screening agents;
propenetrating agents;
antioxidants;
and synthetic polymers.

Of course, those skilled in the art will take care to choose the possible compound(s) to be added to these compositions in such a way that the advantageous properties intrinsically associated with the present invention are not, or are not substantially, altered by the envisaged addition.

The compositions according to the invention are more particularly suited for treating the skin and the mucous membranes, and may be provided in the form of ointments, creams, milks, salves, powders, impregnated pads, syndets, solutions, gels, sprays, foams, suspensions, lotions, sticks, shampoos, pledgets or washing bases. They may also be provided in the form of suspensions of lipid or polymer vesicles or nanospheres or microspheres or polymer patches and hydrogels to allow controlled release. This topical-application composition may be provided in anhydrous form, in aqueous form or in the form of an emulsion.

The compositions according to the invention showing improved penetration are preferably administered in the form of a sprayable composition. In order to be sprayable, the compositions according to the invention will preferably have a viscosity of less than 50 centistokes, and more preferably less than 10 centistokes.

The spray can be obtained by conventional formulation means known to those skilled in the art. For example, the composition may be sprayed by means of a mechanical spraying device which pumps the composition from a container, bottle or equivalent. The composition passes through a nozzle which can be aimed directly at the desired site of application. The nozzle can be selected so as to apply the composition in the form of a vaporization or of a jet of droplets, according to techniques known to those skilled in the art. According to the pharmaceutical active agent selected, the spraying mechanism must be capable of always delivering the same amount of active agent. The mechanisms for controlling the amount of composition to be delivered by the spray are also known to those skilled in the art. For example, the amount of propellant gas can be calculated so as to propel the exact amount of product desired.

Preferably, for the compositions according to the invention, a dosing spray bottle, for which the application area and dose characteristics are controlled and reproducible, will be used. For example, the spray device used consists of a bottle equipped with a 25 μl dosing valve.

The present invention also features the administration of a composition according thereto, in a medicinal product suited for a regime or regimen for treating:
dermatological conditions associated with a keratinization disorder relating to differentiation and to proliferation, in particular common acne, comedo-type acne, polymorphic acne, rosacea, nodulocystic acne, acne conglobata, senile acne, and secondary acne such as solar, drug-related or occupational acne,
ichthyoses, ichthyosiform conditions, Darrier's disease, palmoplantar keratoderma, leukoplakia and leukoplakiform conditions, and cutaneous or mucosal (oral) lichen,
dermatological conditions with an inflammatory immunoallergic component, with or without a cell proliferation disorder, in particular cutaneous, mucosal or ungual psoriasis, psoriatic rheumatism, cutaneous atopy, such as eczema, respiratory atopy or gingival hypertrophy,
benign or malignant dermal or epidermal proliferations, of viral or non-viral origin, in particular common warts, flat warts, epidermodysplasia verruciformis, oral or florid papillomatoses, and T lymphoma,
proliferations which may be induced by ultraviolet light, in particular basal cell epithelioma and spinocellular epithelioma,
precancerous skin lesions, in particular keratoacanthomas,
immune dermatoses, in particular lupus erythematous,
bullous immune diseases,
collagen diseases, in particular scleroderma,
dermatological or systemic conditions with an immunological component,
skin disorders due to exposure to UV radiation, or light-induced or chronological ageing of the skin, or actinic keratoses and pigmentations, or any pathologies associated with chronological or actinic ageing, in particular xerosis,
sebaceous function disorders, in particular hyperseborrhoea acne or simple seborrhoea or seborrhoeic dermatitis,
cicatrization disorders or stretch marks,
pigmentation disorders, such as hyperpigmentation, melasma, hypopigmentation or vitiligo,
lipid metabolism conditions, such as obesity, hyperlipidemia, non-insulin-dependant diabetes or syndrome X,
inflammatory conditions such as arthritis,
cancerous or precancerous states,
alopecia of various origins, in particular alopecia caused by chemotherapy or radiation,
immune system disorders, such as asthma, type 1 diabetes mellitus, multiple sclerosis or other selective dysfunctions of the immune system, or
cardiovascular system conditions such as arteriosclerosis or hypertension.

In a preferred embodiment of use of the composition, said composition will contain 0.05% of clobetasol 17-propionate and will be used for producing a medicinal product suited to treat psoriasis.

This invention also features a process for improving the penetration of an active agent, wherein a composition comprising the following, formulated into a pharmaceutically acceptable alcoholic vehicle, is topically applied onto the skin:
a) a therapeutically effective amount of a pharmaceutical active agent,
b) at least one volatile silicone, and
c) a non-volatile oily phase,
   said composition being applied by spraying.

Preferably, the process will be such that the active agent is clobetasol 17-propionate, the volatile silicone is hexamethyldisiloxane and the non-volatile oily phase is paraffin oil.

In one embodiment, the process will be such that the composition also comprises a silicone gum.

Indeed, it has been surprisingly discovered that the penetration of an active agent through the skin is improved by the compositions according to the invention. The expression “improvement in penetration into the skin” means a significant increase in penetration into the skin of at least a factor of 2, compared to the known formulations on the market. The penetration of the active agent is measured according to the protocol described in Example 7.

The following examples are examples of formulation of the compositions according to the invention and results of penetration into the skin, and also results of acceptability and of tolerance of the composition according to the invention, compared to existing formulas. It should be understood that same are intended only as illustrative and in nowise limitative. In said examples to follow, all parts and percentages are given by weight, unless otherwise indicated.

### EXAMPLE 1

### Composition

The formulation is obtained by mixing the various compounds mentioned below until a homogeneous and clear solution is obtained.

| Ingredients | Function | Spray A |
|---|---|---|
| Clobetasol | Active agent | 0.05% |
| 17-propionate | | |
| Hexamethyldisiloxane | Volatile silicone | 60.0% |
| Paraffin oil | Non-volatile oily phase | 10.0% |
| Absolute ethanol | Solvent: excipient | qs 100% |

### EXAMPLE 2

### Composition

The procedure used is the same as that in Example 1.

| Ingredients | Function | Spray B |
|---|---|---|
| Clobetasol | Active agent | 0.05% |
| 17-propionate | | |
| Hexamethyldisiloxane | Volatile silicone | 59.4% |
| Silicone gum | Silicone gum | 0.6% |
| Paraffin oil | Non-volatile oily phase | 10.0% |
| Absolute ethanol | Solvent | qs 100% |

### EXAMPLE 3

### Composition

The procedure used is the same as that in Example 1.

| Ingredients | Function | Spray C |
|---|---|---|
| Clobetasol | Active agent | 0.05% |
| 17-propionate | | |
| Hexamethyldisiloxane | Volatile silicone | 59.4% |
| Silicone gum | Silicone gum | 0.6% |
| Paraffin oil | Non-volatile oily phase | 10.0% |
| Oleic acid | Propenetrating agent | 5.0% |
| Butylhydroxytoluene | Antioxidant | 0.05% |
| (BHT) | | |
| Absolute ethanol | Solvent | qs 100% |

### EXAMPLE 4

### Composition

The procedure used is the same as that in Example 1.

| Ingredients | Function | Spray D |
|---|---|---|
| Amorolfine HCl | Active agent | 5.0% |
| Hexamethyldisiloxane | Volatile silicone | 59.4% |
| Silicone gum | Silicone gum | 0.6% |
| Isodecyl oleate | Non-volatile oily phase | 5.0% |
| Urea | Propenetrating agent | 5.0 |
| Absolute ethanol | Solvent | qs 100% |

### EXAMPLE 5

### Composition

The procedure used is the same as that in Example 1.

| Ingredients | Function | Spray E |
|---|---|---|
| Calcipotriol | Active agent | 0.005% |
| Hexamethyldisiloxane | Volatile silicone | 59.4% |
| Silicone gum | Silicone gum | 0.6% |
| Paraffin oil | Non-volatile oily phase | 10.0% |
| Absolute ethanol | Solvent | qs 100% |

### EXAMPLE 6

### Composition

The procedure used is the same as that in Example 1.

| Ingredients | Function | Spray F |
|---|---|---|
| Clindamycin | Active agent | 1.0% |
| Hexamethyldisiloxane | Volatile silicone | 59.4% |
| Silicone gum | Silicone gum | 0.6% |
| Isopropyl myristate | Non-volatile oily phase | 5.0% |
| Absolute ethanol | Solvent | qs 100% |

### EXAMPLE 7

### Study of the Release/Penetration in Vitro, on Human Skin, of the Active Agent Clobetasol 17-Propionate Contained in 4 Different Formulations, Three of Which are Sprayable

The first objective is to quantify the penetration into the skin of the active agent formulated in various formulations, in vitro, on human skin, after 16 hours of application.

The second objective is to evaluate the influence of the formulation on the kinetics of penetration of the active agent through and into the skin. For this purpose, a shorter application time was tested: 4 hours.

Formulations Tested:
Temovate® emollient cream containing 0.05% (w/w) of clobetasol 17-propionate
Spray A
Spray B
Spray C

The Temovate® emollient cream is sold by GLAXOSMITHKLINE.

The exact compositions of the three compositions are given in Table A below, and correspond to Examples 1, 2 and 3 of the present invention.

TABLE A

| Ingredients | Function | Spray A | Spray B | Spray C |
|---|---|---|---|---|
| Clobetasol | Active agent | 0.05% | 0.05% | 0.05% |
| 17-propionate | | | | |
| Hexamethyldisiloxane | Volatile | 60.0% | 59.4% | 59.4% |
| | silicone | | | |
| Silicone gum | Silicone gum | / | 0.6% | 0.6% |
| Paraffin oil | Occlusive | 10.0% | 10.0% | 10.0% |
| | agent | | | |
| Oleic acid | Propenetrat- | / | / | 5.0% |
| | ing agent | | | |
| BHT | Antioxidant | / | / | 0.05% |
| Absolute ethanol | Solvent | qs 100% | qs 100% | qs 100% |

Experimental Conditions:

Percutaneous absorption is evaluated by means of diffusion cells consisting of 2 compartments separated by human skin. The formulations were applied without occlusion.

Two application times were tested: 4 and 16 hours.

The formulations were applied at a rate of 20 mg of formulation per 2 cm² (i.e., 10 micrograms of clobetasol 17-propionate).

Throughout the duration of the study, the dermis is in contact with a recipient liquid which is not renewed as a function of time (static mode).

For each application time, six different experiments were carried out with six samples of skin originating from six different donors.

At the end of the application period, the surface excess is removed and the distribution of the clobetasol 17-propionate is quantified in the various skin compartments and in the recipient liquid. The concentrations of clobetasol 17-propionate were quantified using an HPLC/MS/MS method conventionally known to those skilled in the art (LQ: 10 ng.mL⁻¹).

The spray formulas were applied using a spray bottle equipped with a 25 μl dosing valve.

The experimental results show that, whatever the formulation tested, the active agent is distributed mainly in the skin (epidermis, including strateum corneum, and dermis). The total amounts penetrated (stratum corneum+epidermis+dermis+recipient liquid) are:

| | | Application |
|---|---|---|
| | Application time: | time: |
| | 4 hours | 16 hours |
| Temovate ® emollient cream | | |
| Total amount having penetrated | | |
| μg | 0.52 ± 13 μg | 0.67 ± 0.08 μg |
| % dose applied | 5% | 7% |
| Spray A | | |
| Total amount having penetrated | | |
| μg | 0.57 ± 0.23 μg | 1.35 ± 0.45 μg |
| % dose applied | 7% | 17% |
| Spray B | | |
| Total amount having penetrated | | |
| μg | 0.96 ± 0.29 μg | 1.31 ± 0.35 μg |
| % dose applied | 11% | 15% |
| Spray C | | |
| Total amount having penetrated | | |
| μg | 1.01 ± 37 μg | 1.49 ± 0.39 μg |
| % dose applied | 10% | 14% |

Results:

The results show here that Spray A shows a greater than two-fold increase in penetration of the active agent after 16 hours, compared with the formula of the already existing Temovate® emollient cream, although this composition, formula A, according to the invention contains neither propenetrating compound nor occlusive agent.

Formulas B and C, although they contain substantive silicone gum, allow good release of the active agent, therefore also resulting in good penetration of the active agent.

The measurement carried out at time 4 hours makes it possible to evaluate the influence of the excipients on the rapidity of penetration of the active agent in the various spray forms. The results show that the addition of silicone gum to the Spray B and C compositions increases the rapidity of penetration compared with the Spray A formula which does not contain any.

Moreover, it may be noted that the addition within Spray C of a propenetrating agent such as oleic acid does not significantly modify the penetration of the active agent within one of the spray formulas according to the invention.

By way of indication, the spray formulations according to the invention were also compared to a lotion formulation containing clobetasol 17-propionate as described in EP-0-832,647 and comprising from 40 to 50% of propenetrating glycol. The result for penetration of the active agent within this formula is as follows:

| Lotion containing clobetasol 17-propionate | |
|---|---|
| Total amount having penetrated* | |
| μg | 0.60 ± 0.07 μg |
| % dose applied | 12% |

*the surface area of application of the products in this experiment is 1 cm²

The lotion increases the penetration of the active agent by a factor of greater than 2, after 16 hours of application, compared with the already existing Temovate® emollient cream formula. This result therefore indicates that the compositions according to the invention, even in the absence of propenetrating compounds, make it possible to obtain a significant improvement in penetration of an active agent compared with existing formulas, or a penetration similar to compositions having a high percentage of propenetrating compounds.

The spray formulas as described therefore make it possible to do without the use of glycols, without decreasing skin penetration, and therefore show an additional advantage in terms of non-irritant potential versus the compositions comprising a high content of glycol.

### EXAMPLE 8

### Evaluation of the Cosmetic Acceptability of the Sprays According to the Invention

The objective of this study was to evaluate the cosmetic qualities of a sprayable composition in a usage test, after 5 days of application to target lesions of patients (15 male or female individuals, 18 to 60 years old, exhibiting at least 3 psoriatic plaques showing slight to moderate psoriasis). The composition tested here is the spray B formula of Example 2.

In addition, this study should make it possible to situate the formulation of the sprayable composition with respect to the vehicles of products already known (cream and lotion).

For the individuals, the sprayable composition stands out significantly (p<0.05) from the other 2 products for:
ease of application,
rapidity of drying,
rapidity of penetration, and therefore
possibility of getting dressed more rapidly,
lack of greasy or sticky feeling to the skin.

The individuals did not find that the sprayable composition spilled over too much onto the non-affected skin, compared to the other 2 products (p<0.05); moreover, it appeared to them to be less liquid than the lotion (p<0.05). It appeared to them to be more practical for treating areas difficult to reach, such as the back, compared to the lotion (p<0.05), but not compared to the cream.

In terms of approval of the product, 74% of the individuals gave a favorable opinion (good or excellent) of the sprayable composition, which stands out clearly from the lotion (54%), but less clearly from the cream (67%). No patient gave an unfavorable opinion of the spray, versus 7% for the lotion and 14% for the cream.

### EXAMPLE 9

### Evaluation of the Local Tolerance, the Moisturizing Potential and the Effect on the Barrier Function and the pH of the Skin

Another study was carried out with the sprays of qualitative and quantitative compositions below, with the objective of evaluating the local tolerance, the moisturizing potential, and the effect on the barrier function and the pH of the skin of two sprayable vehicles applied to the forearms of normal individuals.

| Ingredients | Function | Spray D | Spray E |
|---|---|---|---|
| Hexamethyldisiloxane | Volatile silicone | 59.4% | 59.4% |
| Silicone gum | Silicone gum | 0.6% | 0.6% |
| Paraffin oil | Occlusive agent | / | 10.0% |
| Absolute ethanol | Solvent | qs 100% | qs 100% |

The study was as follows: a local tolerance test lasting 21 days (plus an inclusion visit) was carried out on 12 normal individuals, male or female, 18 to 50 years old.

Three areas were delimited: an area on each forearm in the region of the fold of the elbow, and an untreated area on the right forearm in the region of the wrist.

The individuals were given the 2 test products, on the forearms, according to a pre-established randomization plan, once a day, every day for 21 days, in a proportion of 50 μl per area.

Clinical evaluations of tolerance (erythema, desquamation, pruritus and burning), were carried out on D0, D7, D14 and D21. A marking scale of 0 to 3 was used to evaluate the erythema and the desquamation (0=absent, 1=slight, 2=moderate and 3=severe). A marking scale of 0 to 9 was used to evaluate sensations of pruritus and burning sensations (0=absent, 1-2-3=slight, 4-5-6=moderate and 7-8-9=severe).

Corneometry, colorimetry and pH measurements were taken on D0, D14 and D21, along with transepidermal water loss (TEWL) measurements. All these measurements were taken before the application of the products, after an acclimatization period of 15 minutes for all the individuals.

Evaluation Criteria:

Main Criterion:
Clinical evaluation of signs of irritation: total erythema and desquamation score on a marking scale of 0 to 6.
Clinical evaluation of the symptoms of irritation: total pruritus and burning score on a marking scale of 0 to 18.
Tolerance.

Secondary Criteria:
Colorimetry measurements, parameter a* (chromaticity variable, red-green axis).
Biophysical measurements: corneometry (electrical capacitance in arbitrary units), pH and TEWL (in g/m²/h).

Statistical Analyses:

For the tolerance test, an area under the curve was calculated, if appropriate, in order to quantify an overall effect of the treatments over the evaluation period. The parameters were adjusted beforehand with their initial value, before application. The TEWL variable was converted to natural logarithm in order to normalize the distribution and stabilize the variance.

A student's t test analysis of variance, comprising the factors, individual, area, product monitored, was used to compare the AUCs (the threshold of 0.05 was used to reach a conclusion of significance).

Results:

Main Criteria:

Signs and Symptoms of Irritation:

The irritation scores were often zero. No statistical analysis was therefore performed. Only one individual gave a non-zero score. The marks were, however, very close to 0 (total score of 1).

Total Score Over the 4 Visits:

| | Spray D | Spray E | Untreated area |
|---|---|---|---|
| Erythema | 0 | 0 | 0 |
| Desquamation | 0 | 0 | 0 |
| Pruritus | 0 | 0 | 0 |
| Burning | 1 | 0 | 0 |

Tolerance:

The tolerance was monitored through adverse events (AE). Nine individuals (75.0%) out of 12 presented an AE. Since these AEs were not related to the treatment, the tolerance to the products was judged to be very good.

Secondary Criteria:

Evolution of Parameter a*: Colorimetry (n=12; Mean±Sem):

| | D0 | D14 | D21 |
|---|---|---|---|
| Spray D | 8.38 ± 0.41 | 8.73 ± 0.43 | 8.81 ± 0.45 |
| Spray E | 8.48 ± 0.36 | 8.08 ± 0.38 | 8.45 ± 0.43 |
| Untreated area | 7.71 ± 0.54 | 7.86 ± 0.56 | 7.96 ± 0.53 |

There is no significant difference from the two formulations tested for the parameter a* which makes it possible to more precisely evaluate the erythema and therefore the irritation. This should be linked together with the lack of irritant capacity observed from a clinical point of view.

Evolution of the TEWL (n=12; Mean±Sem):

| | D0 | D14 | D21 |
|---|---|---|---|
| Spray D | 63.64 ± 2.26 | 59.69 ± 1.58 | 61.58 ± 1.61 |
| Spray E | 61.33 ± 2.06 | 62.42 ± 1.87 | 64.61 ± 2.39 |
| Untreated area | 60.39 ± 1.99 | 59.78 ± 2.08 | 61.72 ± 2.06 |

The two formulations tested have no significant effects on the transepidermal waterloss (TEWL), which reflects the degree of functional integrity of the cornefied layer. This confirms the lack of irritant potential of the two formulations.

Evolution of the pH (n=12; Mean±Sem):

| | D0 | D14 | D21 |
|---|---|---|---|
| Spray D | 5.34 ± 0.18 | 4.98 ± 0.20 | 4.96 ± 0.09 |
| Spray E | 5.29 ± 0.17 | 4.98 ± 0.13 | 5.04 ± 0.16 |
| Untreated area | 5.17 ± 0.22 | 4.98 ± 0.14 | 4.91 ± 0.11 |

There is no significant difference between the two formulations tested for the pH. They therefore preserve the acid physiological pH of the skin, which is one of the elements of the barrier function of the skin.

Conclusion:

This study shows the good tolerance of the spray vehicles despite the high ethanol content of 30%.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A sprayable, topically applicable pharmaceutical composition comprising:
a) a therapeutically effective amount of at least one pharmaceutical active agent,
b) at least one volatile silicone, and
c) a non-volatile oily phase, formulated into d) a sprayable, topically applicable, pharmaceutically acceptable alcoholic vehicle therefor.

2. The sprayable pharmaceutical composition as defined by claim 1, comprising:
a) from 0.0001 to 20% by weight of the pharmaceutical active agent,
b) from 25 to 95% by weight of volatile silicone, and
c) from 1 to 50% by weight of non-volatile oily phase.

3. The sprayable pharmaceutical composition as defined by claim 1, said at least one pharmaceutical active agent comprising a corticoid compound.

4. The sprayable pharmaceutical composition as defined by claim 3, said at least one pharmaceutical active agent comprising clobetasol 17-propionate.

5. The sprayable pharmaceutical composition as defined by claim 4, comprising from 0.01% to 2% by weight of clobetasol 17-propionate.

6. The sprayable pharmaceutical composition as defined by claim 1, said at least one volatile silicone comprising a polydimethylcyclosiloxane and/or a low molecular weight linear polysiloxane.

7. The sprayable pharmaceutical composition as defined by claim 6, comprising a linear hexamethyldisiloxane polysiloxane.

8. The sprayable pharmaceutical composition as defined by claim 7, comprising from 55 to 65% by weight of hexamethyldisiloxane.

9. The sprayable pharmaceutical composition as defined by claim 1, said non-volatile oily phase comprising paraffin oil.

10. The sprayable pharmaceutical composition as defined by claim 9, comprising 5 to 15% of paraffin oil.

11. The sprayable pharmaceutical composition as defined by claim 1, further comprising a silicone gum.

12. The sprayable pharmaceutical composition as defined by claim 1, having a viscosity of less than 50 centistokes.

13. The sprayable pharmaceutical composition as defined by claim 1, comprising:
a) 0.05% of clobetasol 17-propionate,
b) 60% of hexamethyldisiloxane,
c) 10% of paraffin oil,
d) 29.95% of ethanol.

14. The sprayable pharmaceutical composition as defined by claim 1, comprising:
a) 0.05% of clobetasol 17-propionate,
b) 59.4% of hexamethyldisiloxane,
c) 0.6% of silicone gum,
d) 10% of paraffin oil,
e) 29.95% of ethanol.

15. The sprayable pharmaceutical composition as defined by claim 1, said at least one pharmaceutical active agent being selected from the group consisting of agents for modulating the differentiation and/or proliferation and/or pigmentation of the skin; anti-bacterial agents; anti-parasitic agents; anti-fungal agents; polyene compounds; allylamine compounds; pyridinone compounds; morpholine compounds; steroidal anti-inflammatories; non-steroidal anti-inflammatories; anaesthetic compounds; anti-pruriginous agents; anti-viral agents; keratolytic agents; free-radical scavengers; anti-seborrhoeic agents; anti-dandruff agents; anti-acne agents; anti-metabolites; agents for combating hair loss; antiseptics; hormones; peptides; and mixtures thereof.

16. A regime or regimen for the treatment of psoriasis, comprising spraying onto the affected skin area of an individual in need of such treatment, a thus effective amount of the sprayable pharmaceutical composition as defined by claim 1.

17. A regime or regimen for the treatment:
of dermatological conditions associated with a keratinization differentiation or proliferation disorder;
of keratinization disorders;
of precancerous skin lesions;
of dermatological conditions associated with a keratinization disorder with an inflammatory and/or immunoallergic component;
of ichthyoses, ichthyosiform conditions, Darrier's disease, palmoplantar keratoderma, leukoplakia and leukoplakiform conditions, and cutaneous or mucosal (oral) lichen,
of inflammatory conditions;
of cardiovascular disorders;
of inflammatory skin conditions not exhibiting a keratinization disorder;
of pigmentation disorders;
of dermal or epidermal proliferations;
of alopecia;
of dermatological disorders, bullous dermatosis and collagen diseases;
of lipid metabolism disorders;
of the signs of skin aging, whether photoinduced or chronological, or for reducing actinic pigmentations and keratoses, or any skin pathologies associated with chronological or actinic aging;
of cicatrization disorders or stretch marks;
of cancerous or precancerous conditions;
of sebaceous function disorders, acne hyperseborrhoea or ordinary seborrhoea or seborrhoeic dermatitis;
of immune system disorders;
of dermatological conditions with an immunological component, comprising administering to an individual in need of such treatment, a thus effective amount of the pharmaceutical composition as defined by claim 1.

18. A process for improving the penetration of at least one pharmaceutical active agent into the skin, comprising formulating at least one volatile silicone, a non-volatile oily phase and a pharmaceutically acceptable alcoholic vehicle herewith, and spraying said formulation onto the skin of an individual in need of such treatment.

19. The process as defined by claim 18, said at least one pharmaceutical active agent comprising clobetasol 17-propionate.

20. The process as defined by claim 18, said formulation further comprising a silicone gum.
